(19) United States
(12) Patent Application Publication
(10) Pub. No.: US 2014/0343645 A1
(43) Pub. Date: Nov. 20, 2014

(54) ELECTRICAL STIMULATION LEADS AND SYSTEMS WITH ANCHORING UNITS AND METHODS OF MAKING AND USING

(71) Applicant: BOSTON SCIENTIFIC NEUROMODULATION CORPORATION, Valencia, CA (US)

(72) Inventor: David Ernest Wechter, Santa Clarita, CA (US)

(73) Assignee: BOSTON SCIENTIFIC NEUROMODULATION CORPORATION, Valencia, CA (US)

(21) Appl. No.: 14/263,922

(22) Filed: Apr. 28, 2014

Related U.S. Application Data
(60) Provisional application No. 61/823,240, filed on May 14, 2013.

(51) Int. Cl. A61N 1/05; A61N 1/375

(52) U.S. Cl. CPC A61N 1/05; A61N 1/3752
USPC 607/116; 29/854

(57) ABSTRACT

An electrical stimulation lead includes a lead body having a distal end portion, a proximal end portion, and a longitudinal length; electrodes disposed along the distal end portion of the lead body; terminals disposed along the proximal end portion of the lead body; conductors electrically coupling the terminals to the electrodes; and at least one anchoring unit disposed along the distal end portion of the lead body. Each anchoring unit includes a cylindrical lead attachment element defining a central lumen within which a portion of the lead is received, and at least one anchoring element this is disposed over the lead attachment element and extends away from the lead attachment element and is configured and arranged for contact with patient tissue to anchor the lead within the patient tissue.

### CROSS-REFERENCE TO RELATED APPLICATIONS

This application claims the benefit under 35 U.S.C. §119(e) of U.S. Provisional Patent Application Ser. No. 61/823,240, filed May 14, 2013, which is incorporated herein by reference.

### FIELD

The present invention, is directed to the area of implantable electrical stimulation systems and methods of making and using the systems, and in particular implantable electrical stimulation leads having anchoring units and methods of making and using the leads.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

One concern regarding implanted leads is lead migration. This may occur over time and result, in movement of the lead away from the desired tissue for stimulation so as to reduce the effectiveness of therapeutic treatment.

### BRIEF SUMMARY

One embodiment is an electrical stimulation lead that includes a lead body having a distal end portion, a proximal end portion, and a longitudinal length; electrodes disposed along the distal end portion of the lead body; terminals disposed along the proximal end portion of the lead body; conductors electrically coupling the terminals to the electrodes; and at least one anchoring unit disposed along the distal end portion of the lead body. Each anchoring unit includes a cylindrical lead attachment element defining a central lumen within which a portion of the lead is received, and at least one anchoring element this is disposed over the lead attachment element and extends away from the lead attachment element and is configured and arranged for contact with patient tissue to anchor the lead within the patient tissue.

Another embodiment is an electrical stimulation system that includes the electrical stimulation lead described above and a control module coupleable to the electrical stimulation lead.

Yet another embodiment is a method of making an electrical stimulation lead. The method includes forming an electrical stimulation lead, including a lead body having a distal end portion, a proximal end portion, and a longitudinal length, electrodes disposed along the distal end portion of the lead body, terminals disposed along the proximal end portion of the lead body, and conductors electrically coupling each of the plurality of terminals to at least one of the plurality of electrodes; and disposing at least one anchoring unit along the distal end portion of the lead body. Each anchoring unit includes a cylindrical lead attachment element defining a central lumen within which a portion of the lead is received, and at least one at least one anchoring element this is disposed over the lead attachment clement and extends away from the lead attachment element and is configured and arranged for contact with patient tissue to anchor the lead within the patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a lead electrically coupled to a control module, according to the invention;

FIG. 2A is a schematic side view of one embodiment of the control module of FIG. 1 configured and arranged to electrically couple to an elongated device, according to the invention;

FIG. 2B is a schematic side view of one embodiment of a lead extension configured and arranged to electrically couple the elongated device of FIG. 2A to the control module of FIG. 1, according to the invention;

FIG. 3 is a schematic perspective view of one embodiment of a lead anchoring unit, according to the invention;

FIG. 4 is a schematic perspective view of a second embodiment of a lead anchoring unit, according to the invention;

FIG. 5 is a schematic perspective view of a third embodiment of a lead anchoring unit, according to the invention;

FIG. 6A is a schematic side view of one embodiment of a distal portion of a lead with lead anchoring units disposed thereon, according to the invention;

FIG. 6B is a schematic cross-sectional view of a portion of the lead body and one of the lead anchoring units of FIG. 6A, according to the invention;

FIG. 7 is a schematic side view of another embodiment of a distal portion of a lead with lead anchoring units disposed thereon, according to the invention; and

FIG. 8 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems, and in particular implantable electrical stimulation leads having anchoring units and methods of making and using the leads.

Suitable implantable electrical stimulation systems include, but are not limited to, at least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example. U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Applications Publication Nos. 2007/0150036 and 2010/0256696, all of which are incorporated by reference.

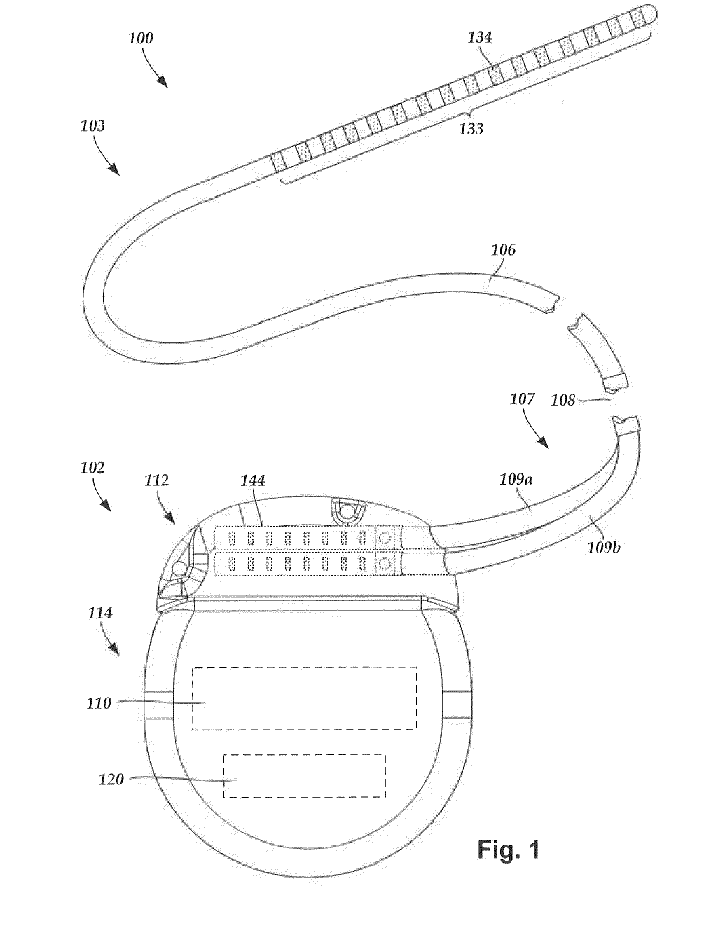

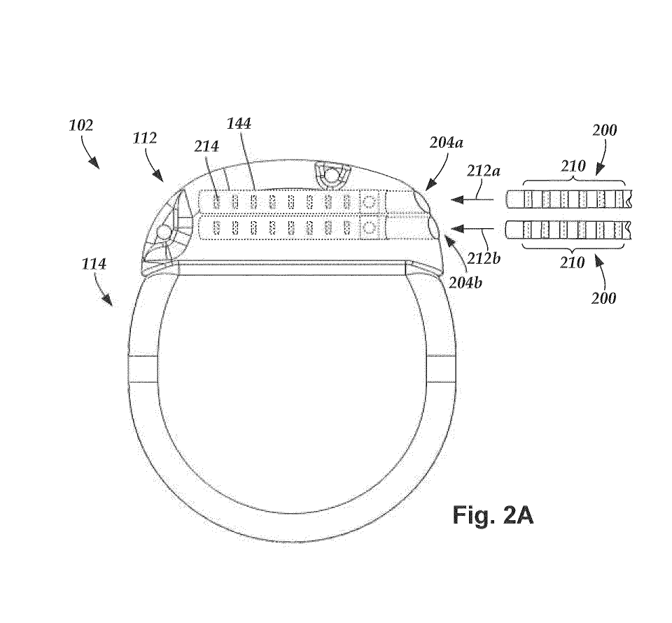

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes one or more lead bodies 106, an array of electrodes 133, such as electrode 134, and an array of terminals (e.g., 210 in FIG. 2A-2B) disposed along the one or more lead bodies 106. In at least some embodiments, the lead is isodiametric along a longitudinal length of the lead body 106.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including one or more of the lead bodies 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the one or more lead bodies 106 are typically disposed in, or separated by, a non-conductive, biocompatible material such, as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 210 in FIGS. 2A-2B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIGS. 2A-2B; and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably scalable at the distal end.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the splitter 107 of FIG. 1, the lead extension 224 of FIG. 2B, an adaptor, or the like or combinations thereof), or a combination thereof.

The control module connector 144 defines at least one port into which a proximal end of the elongated device 200 can be inserted, as shown by directional arrows 212a and 212b. in FIG. 2A (and in other figures), the connector housing 112 is shown having two ports 204a and 204b. The connector housing 112 can define any suitable number of ports including, for example, one, two, three, four, live, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. No. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, the splitter 107, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Lead anchoring units can be attached to the lead to facilitate anchoring the lead into patient tissue. The term “tissue” includes, but is not limited to, muscular tissue, connective tissue, organ tissue, bone, cartilage, nerve tissue, and the like. These lead anchoring units, as opposed to conventional lead anchors, can be delivered with the lead through an introducer during the implantation process. The lead anchoring units include anchoring elements that lodge against patient tissue and prevent or reduce lateral or axial (or both lateral and axial) migration of the lead after implantation. The lead anchoring units can be particularly useful for leads for sacral nerve stimulation, spinal cord stimulation, or the stimulation of other patient tissue and organs.

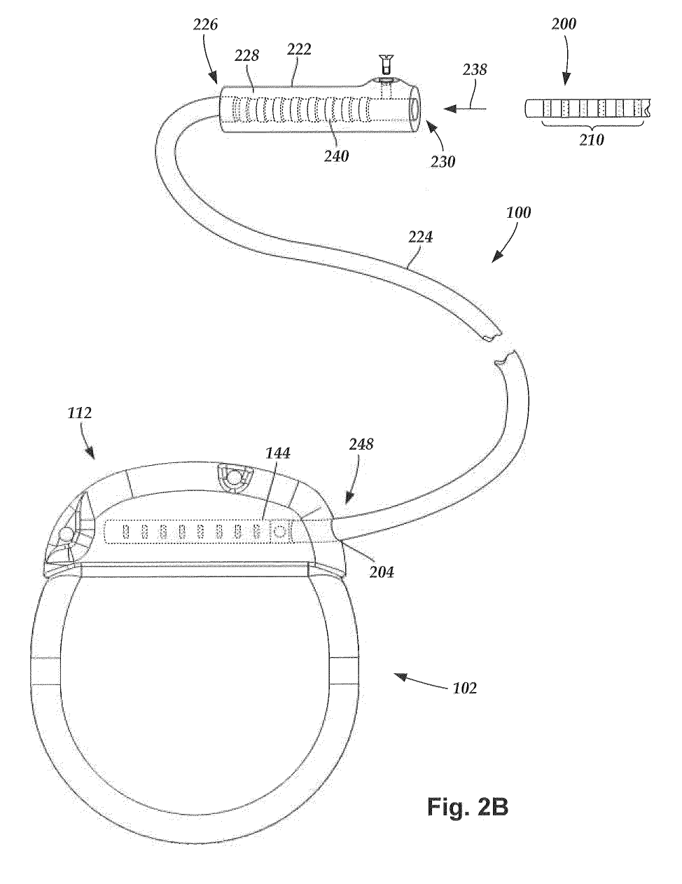

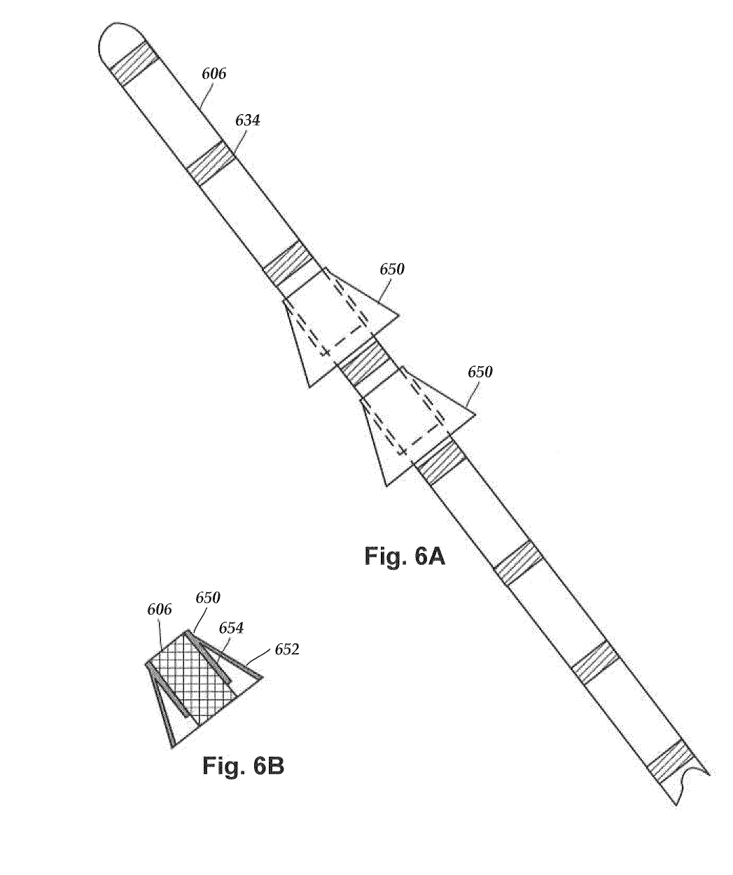

FIG. 3 is a schematic perspective view of one embodiment of a lead anchoring unit 350 that will be disposed along a distal end portion of a lead body (e.g., the lead body 106 as shown in FIG. 1 or the lead body 606 of FIG. 6A). The lead anchoring unit facilitates anchoring the lead body to the surrounding tissue when implanted within a patient's body.

The anchoring unit 350 includes a load attachment element 354 having a tube-shaped (e.g., cylindrical) configuration. As shown, the lead attachment element 354 has a proximal end 351, a distal end 353, and a central lumen 356 extending between the two ends 351, 353. The central lumen 356 may be referred to as an “attachment lumen 356”. The attachment lumen 356 is employed to receive at least a portion of the lead body of a lead. In at least some embodiments, the lead attachment element 354 has a circular cross-section. However, the lead attachment element 354 can be formed of any other suitable shape, including shapes having an elliptical, rectangular, polygonal, irregular, or any other suitable lateral cross-section. The lead attachment element 354 can have a uniform lateral cross section along its entire length or a varying lateral cross-section along its length. In at least some embodiments, the cross-section and dimensions of the lead attachment element 354 are dictated by the configuration of the lead body. In at least some embodiments, the outer diameter of the lead body may be slightly larger than the diameter of the attachment lumen 356 so that the lead attachment element fits snuggly on the lead body.

The anchoring unit 350 includes at least one anchoring element 352 coupled to the lead attachment element 354. Although the coupling may occur anywhere along the lead attachment element 354, in the illustrated embodiment, the anchoring element is coupled to the lead attachment element at, or immediately adjacent to, its distal end 353.

In the illustrated embodiment, the anchoring element 352 includes a cone that extends over the lead attachment element 354. In at least some embodiments, the cone is longer than the lead attachment element 354 so that the cone extends over, and beyond, the lead attachment element 354. In other embodiments, the cone may be shorter than the lead attachment element and only extend over a portion the lead attachment element. The anchoring element 352 may have any other suitable shape to interact with patient tissue to anchor the lead to the tissue.

In at least some embodiments, an interior surface 355 of the lead attachment element 354 may be patterned to assist in maintaining the position of the lead anchoring unit on the lead. The pattern may be regular or irregular and may include features, such as but not limited to, surface roughening, cutouts, grooves, regular or irregular shapes, or the like. In at least some embodiments, an outer surface of the lead body 106 may be patterned. The pattern may be regular or irregular and may include features, such as, but not limited to, surface roughening, cutouts, grooves, regular or irregular shapes, or the like, in some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body can be generated so that the two patterns interlock with each other.

The patterning described above may be formed using any suitable method including, but not limited to, ablation (e.g., RF or laser ablation), grinding, knurling, chemical etching, or the like. The patterning may be made on the spacers (i.e., between adjacent or consecutive electrodes) of the lead body 106.

The anchoring unit 350 may form a friction fit with the lead body to hold the anchoring unit in place. Additionally and alternatively, an adhesive, such as a silicone adhesive, may be employed to bond the anchoring unit 350 to the lead body.

The anchoring unit 350 may be formed of any suitable material, such as any suitable biocompatible material including, but not limited to, metals, polymers, alloys, or the like. In at least some embodiments, the anchoring unit 350 is formed of silicone, polyurethane, or the like. In some embodiments, the material that is used has a stiffness that is sufficient to anchor the lead body to the surrounding tissue, while also having sufficient flexibility to reduce, or in some cases avoid, damage or injury to the tissue or to facilitate delivery of the lead with the anchoring unit(s) through an introducer.

In particular, the anchoring unit 350 may be configured to facilitate deployment through an introducer, such as a needle or cannula. In at least some embodiments, the anchoring unit 350 is sufficiently pliable so that it can be compressed within an introducer during implantation. When the introducer is removed, the anchoring unit 350 may then expand to anchor the lead body 106 to the tissue.

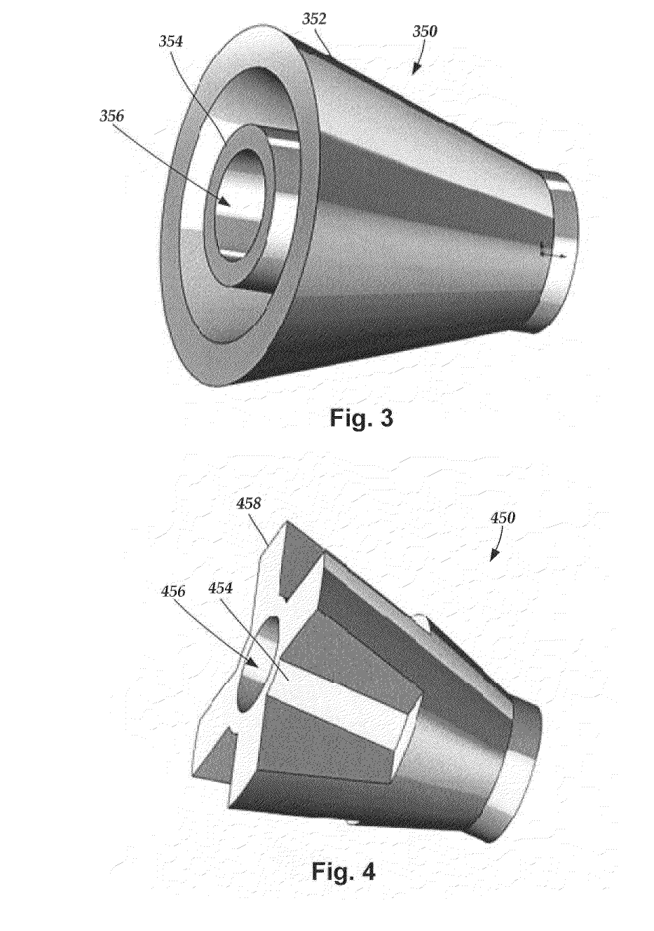

FIG. 4 illustrates a second embodiment of a lead anchoring unit 450 that includes a lead attachment element 454 and at least one anchoring element 458. The lead attachment element 454 receives and attaches to a portion of a lead body 106. The at least one anchoring element 458 anchor the lead body to the patient's tissue.

The lead attachment element 454 has a tube-shaped (e.g., cylindrical) configuration, and includes a proximal end 451, a distal end 453, and a central lumen 456 extending therebetween. The central lumen may also be referred to as “attachment lumen 456”. In at least some embodiments, the lead attachment element 454 has a circular lateral cross-section. However, the lead attachment element 454 can be formed of any other suitable shape, including shapes having an elliptical, rectangular, polygonal irregular, or any other suitable cross-section. The lead attachment element 454 can have a uniform lateral cross-section along its entire length or a varying lateral cross-section along its length. In at least some embodiments, the cross-section and dimensions of the lead attachment element 454 are dictated by the configuration of the lead body. In at least some embodiments, the outer diameter of the lead body may be slightly larger than the diameter of the attachment lumen 456.

In at least some embodiments, an interior surface 455 of the lead attachment element 454 may be patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 106 may be patterned. The pattern may be regular or irregular and may include features, such as, but not limited to, surface roughening, cutouts, grooves, regular or irregular shapes, or the like. In some embodiments, both the interior surface of the lead attachment element and the outer surf ice of the lead body are patterned. The patterning of the lead attachment element and the lead body may be complementary. In at least some embodiments, the pattern on the interior surface of the lead attachment element and the pattern on the exterior surface of the lead body can be generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 106.

The anchoring unit 450 may form a friction fit with the lead body to hold the anchoring unit in place. Additionally and alternatively, an adhesive, such as a silicone adhesive, may be employed to bond the anchoring unit 350 to the lead body.

The anchoring element(s) 458 are disposed around the lead attachment element 454 and extend away from the lead attachment element 454. In the illustrated embodiment, the attachment elements 454 are fins. Any number of fins (or other attachment elements) can be used. The embodiment shown in FIG. 4 includes four fins 454 disposed about the circumference of the lead attachment element 454. The fins 454 shown in FIG. 4 have a trapezoid-shaped configuration, but it will be recognized that the fins 454 can have any suitable shape including, but not limited to, triangular, rectangular, irregular, and the like. Any suitable number of fins may be disposed about the circumference of the lead attachment element 454 including, but not limited to, two, three, four, five, six, seven, eight, or more fins. The fins can be spaced apart uniformly or non-uniformly around the circumference of the lead attachment element 454. In some embodiments, the tins 454 form an angle of ninety degrees with the lead attachment element as illustrated in FIG. 4, but it will be recognized that the fins could extend at a different angle from the lead attachment element (for example, an angle in the range from 30 to 85 degrees).

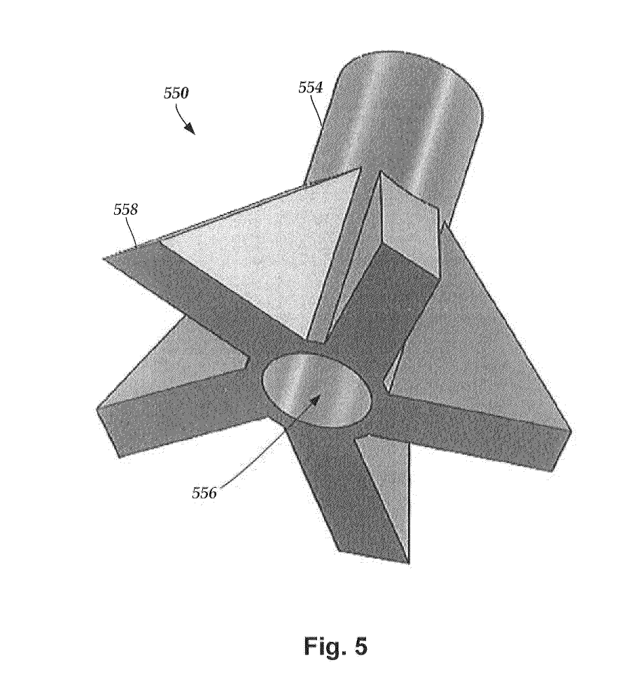

The fins 454 are shown in FIG. 4 as extending along a partial length of the lead attachment element 454, while being disposed about the circumference of the lead attachment element 454. However, in some other embodiments, such as the embodiment shown in FIG. 5, the fins 454 can extend along an entire length of the lead attachment element 454.

FIG. 5 illustrates a third embodiment of a lead anchoring unit 550 that includes a lead attachment element 554 and at least one anchoring element 558. The lead attachment element 554 receives and attaches to a portion of a lead body 106. The at least one anchoring element 558 anchor the lead body to the patient's tissue.

The lead attachment element 554 has a tube-shaped (e.g., cylindrical) configuration, and includes a proximal end 551, a distal end 553, and a central lumen 556 extending therebetween. The central lumen may also be referred to as “attachment lumen 556”. In at least some embodiments, the lead attachment element 554 has a circular lateral cross-section. However, the lead attachment element 554 can be formed of any other suitable shape, including shapes having an elliptical, rectangular, polygonal, irregular, or any other suitable cross-section. The lead attachment element 554 can have a uniform lateral cross-section along its entire length or a varying lateral cross-section along its length. In at least some embodiments, the cross-section and dimensions of the lead attachment element 554 are dictated by the configuration of the lead body. In at least some embodiments, the outer diameter of the lead body may be slightly larger than the diameter of the attachment lumen 556.

In at least some embodiments, an interior surface 555 of the lead attachment element 554 may be patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 106 may be patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body can be generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 106.

The anchoring unit 550 may form a friction fit with the lead body to hold the anchoring unit in place. Additionally and alternatively, on adhesive, such as a silicone adhesive, may be employed to bond the anchoring unit 350 to the lead body.

The anchoring element(s) 558 are disposed around the lead attachment element 554 and extend away from the lead attachment element 554. In the illustrated embodiment, the attachment elements 554 are fins. Any number of fins (or other attachment elements) can be used. The embodiment shown in FIG. 5 includes five fins 554 disposed about the circumference of the lead attachment element 554. The fins 554 shown in FIG. 5 have a triangular-shaped configuration, but it will be recognized that the fins 554 can have any suitable shape including, but not limited to, trapezoidal, rectangular, irregular, and the like. Any suitable number of fins may be disposed about the circumference of the lead attachment element 554 including, but not limited to, two, three, four, five, six, seven, eight, or more fins. The tins can be spaced apart uniformly or non-uniformly around the circumference of the lead attachment element 554. In some embodiments, the fins 554 form an angle of ninety degrees with the lead attachment element as illustrated in FIG. 5, but it will be recognized that the fins could extend at a different angle from the lead attachment element (for example, an angle in the range from 30 to 85 degrees).

The fins 554 are shown in FIG. 5 as extending along a partial length of the lead attachment element 554, while being disposed about the circumference of the lead attachment element 554. However, in some other embodiments, such as the embodiment shown in FIG. 5, the fins 554 can extend along an entire length of the lead attachment element 554.

FIG. 6A is a schematic side view of one embodiment of a distal portion of a lead body 606, with lead anchoring units 650 disposed thereon. The distal portion of the lead body 606 includes multiple electrodes 634 spaced apart in a desired arrangement. In the embodiment of FIG. 6A, eight electrodes 634 are disposed on the lead body 606 in a uniform spaced apart arrangement, however, any suitable number of electrodes 634 can be provided in any suitable arrangement, including but not limited to two, four, sixteen, or more electrodes. Examples of leads are described above with respect to FIGS. 1-2B and the references cited herein.

One or more anchoring units 650 are mounted on the lead body 606. In the illustrated embodiment, the anchoring units 650 are mounted between the electrodes 634, but it will be understood that other embodiments may include some or all of the anchoring units being mounted proximal to, or distal to, the electrodes or any combination thereof.

The anchoring units 650 may be any of the anchoring units describe above including the anchoring units 350, 450, and 550 of FIGS. 3, 4, and 5, respectively. In the embodiment shown in FIG. 6A, two anchoring units 650 are disposed over the lead body 606; however, any suitable number of anchoring units 650 may be used including two, three, four, live, six, seven, eight, nine, ten, or more anchoring units. The anchoring units may be all the same or there may be anchoring units of two or more different types (for examples, a combination of anchoring units 350 and anchoring units 450 or a combination of anchoring units 350 and anchoring units 550 or a combination of anchoring units 550 and anchoring units 450.)

FIG. 6B is a cross-sectional view of a portion of the lead body 606 and one anchoring unit 650. The anchoring unit 650 of FIG. 6B includes an anchoring element 652 and a lead attachment element 654. The lead attachment element 654 has a tubular (e.g., cylindrical) configuration defining a central attachment lumen that receives a portion of the lead body 606. The anchoring element 652 includes a cone 652 that extends over the lead attachment element 654. The cone 652 is longer than the lead attachment element 654, so that the cone extends radially over and beyond the lead attachment element 654. The radially extending cone anchors the lead to the surrounding tissue.

A variety of methods may be employed to attach the anchoring unit 650 to the lead body 606. For example, each individual anchoring unit 650 can be slid onto the lead body 606 to the desired position along the lead body. In some embodiments, the anchoring unit 650 is swelled prior to sliding on the lead body. As an example, a silicone anchoring unit 650 can be treated with a heptane solution to swell the anchoring unit so that it can be slid onto the lead body. As the heptane evaporates, the anchoring unit 650 will return to its original dimensions.

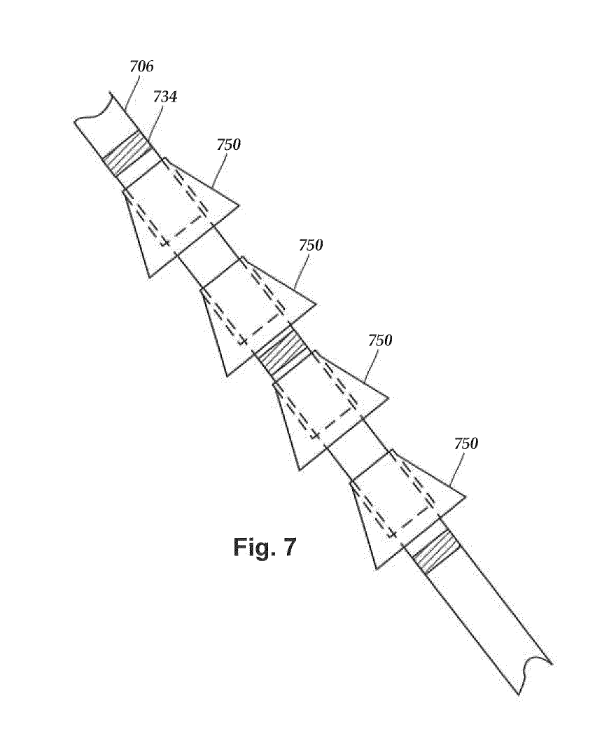

FIG. 7 is a schematic side view of another embodiment of a distal portion of a lead body 706, with lead anchoring units 750 disposed thereon. In contrast to the embodiment shown in FIG. 6A, the embodiment of FIG. 7 includes two anchoring Units 750 disposed between a pair of electrodes 734. In this embodiment, the two anchoring units 750 may overlap or may be spaced apart from each other. Although FIG. 7 shows two anchoring units, any suitable number of anchoring units 750 may be disposed, between a pair of electrodes 734. In some embodiments, two different types of anchoring units 750 are employed to provide enhanced tissue anchorage. As an example, the anchoring unit 350 shown in FIG. 3 may be combined with the anchoring unit 450 shown in FIG. 4 to be disposed between a pair of electrodes 734. Similarly, the pair of anchoring units 350 shown in FIG. 3 and anchoring units 550 shown in FIG. 5 may be disposed between another pair of electrodes 734. Alternatively, the pair of anchoring units 450 shown in FIG. 4 and anchoring units 550 shown in FIG. 5 may be disposed between another pair of electrodes 734.

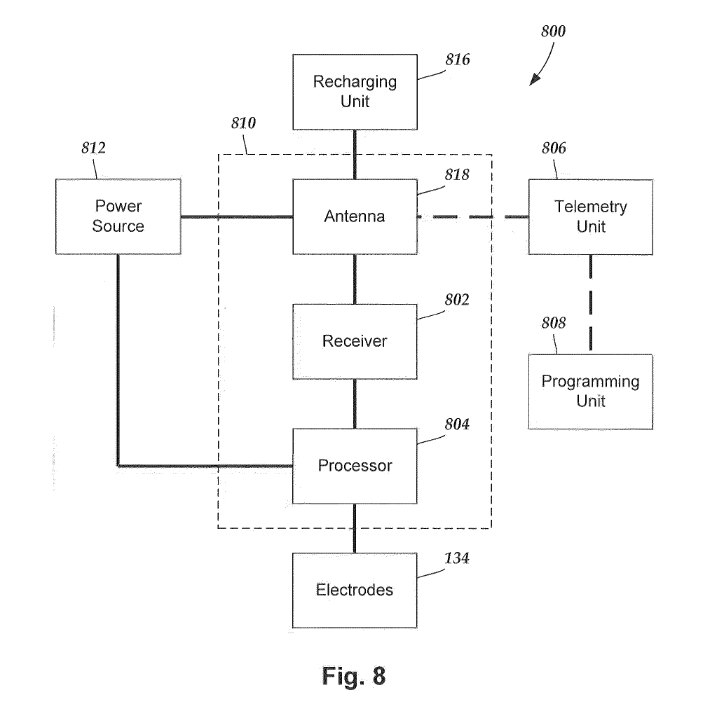

FIG. 8 is a schematic overview of one embodiment of components of an electrical stimulation system 800 including an electronic subassembly 810 disposed within a control module. It will be understood that the electrical stimulation system can include more, fewer, or different components and can have a variety of different configurations including those configurations disclosed in the stimulator references cited herein.

Some of the components (for example, a power source 812, an antenna 818, a receiver 802, and a processor 804) of the electrical stimulation system can be positioned on one or more circuit boards or similar carriers within a sealed housing of an implantable pulse generator, if desired. Any power source 812 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 818 or a secondary antenna. The external power source can be in a device that is mounted on the skin of the user or in a unit that is provided near the user on a permanent or periodic basis.

If the power source 812 is a rechargeable battery, the battery may be recharged using the optional antenna 818, if desired. Power can be provided to the battery for recharging by inductively coupling the battery through the antenna to a recharging unit 816 external to the user. Examples of such arrangements can be found in the references identified above.

In one embodiment, electrical current is emitted by the electrodes 134 on the paddle or lead, body to stimulate nerve fibers, muscle fibers, or other body tissues near the electrical stimulation system. The processor 804 is generally included to control the timing and electrical characteristics of the electrical stimulation system. For example, the processor 804 can, if desired, control one or more of the timing, frequency, strength, duration, and waveform of the pulses. In addition, the processor 804 can select which electrodes can be used to provide stimulation, if desired. In some embodiments, the processor 804 selects which electrode(s) are cathodes and which electrode(s) are anodes. In some embodiments, the processor 804 is used to identify which electrodes provide the must useful stimulation of the desired tissue.

Any processor can be used and can be as simple as an electronic device that, for example, produces pulses at a regular interval or the processor can be capable of receiving and interpreting instructions from an external programming unit 808 that, for example, allows modification of pulse characteristics. In the illustrated embodiment, the processor 804 is coupled to a receiver 802 which, in turn, is coupled to the optional antenna 818. This allows the processor 804 to receive instructions from an external source to, for example, direct the pulse characteristics and the selection of electrodes, if desired.

In one embodiment, the antenna 818 is capable of receiving signals (e.g., RF signals) from an external telemetry unit 806 which is programmed by the programming unit 808. The programming unit 808 can be external to, or part of the telemetry unit 806. The telemetry unit 806 can be a device that is worn on the skin of the user or can be carried by the user and can have a form similar to a pager, cellular phone, or remote control, if desired. As another alternative, the telemetry unit 806 may not be worn or carried by the user but may only be available at a home station or at a clinician's office. The programming unit 808 can be any unit that can provide information to the telemetry unit 806 for transmission to the electrical stimulation system 800. The programming unit 808 can be part of the telemetry unit 806 or can provide signals or information to the telemetry unit 806 via a wireless or wired connection. One example of a suitable programming unit is a computer operated by the user or clinician to send signals to the telemetry unit 806.

The signals sent to the processor 804 via the antenna 818 and the receiver 802 can be used to modify or otherwise direct the operation of the electrical stimulation system. For example, the signals may be used to modify the pulses of the electrical stimulation system such as modifying tare or more of pulse duration, pulse frequency, guise waveform, and pulse strength. The signals may also direct the electrical stimulation system 800 to cease operation, to start operation, to start charging the battery, or to stop charging the battery. In other embodiments, the stimulation system does not include the antenna 818 or receiver 802 and the processor 804 operates as programmed.

Optionally, the electrical stimulation system 800 may include a transmitter (not shown) coupled to the processor 804 and the antenna 818 for transmitting signals back to the telemetry unit 806 or another unit capable of receiving the signals. For example, the electrical stimulation system 800 may transmit signals indicating whether the electrical stimulation system 800 is operating properly or not or indicating when the battery needs to be charged or the level of charge remaining in the battery. The processor 804 may also be capable of transmitting information about the pulse characteristics so that a user or clinician can determine or verity the characteristics.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead, comprising:
a lead body having a distal end portion, a proximal end portion, and a longitudinal length;
a plurality of electrodes disposed along the distal end portion of the lead body;
a plurality of terminals disposed along the proximal end portion of the lead body;
a plurality of conductors, each conductor of the plurality of conductors electrically coupling each of the plurality of terminals to at least one of the plurality of electrodes; and
at least one anchoring unit disposed along the distal end portion of the lead body, the at least one anchoring unit comprising
a cylindrical lead attachment element defining a central lumen within which a portion of the lead is received, and
at least one anchoring element this is disposed over the lead attachment element and extends away from the lead attachment element and is configured and arranged for contact with patient tissue to anchor the lead within the patient tissue.

2. The electrical stimulation lead of claim 1, where the at least one anchoring element is a cone that extends over the lead attachment element.

3. The electrical stimulation lead of claim 2, wherein the cone is attached to the lead attachment clement at one end of the lead attachment element.

4. The electrical stimulation lead of claim 2, wherein the cone has a length longer than a length of the lead attachment element so that the cone extends over, and beyond, the lead, attachment element.

5. The electrical stimulation lead of claim 1, where the at least one anchoring element comprises a plurality of tins, each tin attached to, and extending away from, the lead attachment element.

6. The electrical stimulation lead of claim 5, wherein each fin has a longitudinal length and is attached to the lead attachment element along its entire longitudinal length.

7. The electrical stimulation lead of claim 5, wherein each of the fins has a triangular shape.

8. The electrical stimulation lead of claim 1, wherein the at least one anchoring unit comprises a first anchoring unit disposed between two of the plurality of electrodes.

9. The electrical stimulation lead of claim 1, wherein the plurality of electrodes comprises a first electrode and a second electrode adjacent to the first electrode and the at least one anchoring unit comprises a first anchoring unit and a second anchoring unit, wherein the first and second anchoring units are disposed between the first and second electrode.

10. The electrical stimulation lead of claim 1, wherein the at least one anchoring unit is removably disposed along the distal end portion of the lead body.

11. The electrical stimulation lead of claim 1, wherein at least one region of the lead body is ablated to receive that at least one anchoring unit.

12. The electrical stimulation lead of claim 11, wherein that at least one region is ablated in a pattern and the at least one anchoring unit has a corresponding pattern on an interior surface thereof.

13. An electrical stimulating system comprising:
the electrical stimulation lead of claim 1;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to at least one of the plurality of terminals disposed on the proximal end of the lead body of the electrical stimulation lead.

14. The electrical stimulation system of claim 13, further comprising a lead extension coupleable to both the electrical stimulation lead and the control module.

15. A method of making an electrical stimulation lead, the method comprising:
forming an electrical stimulation lead comprising a lead body having a distal end portion, a proximal end portion, and a longitudinal length, a plurality of electrodes disposed along the distal end portion of the lead body, a plurality of terminals disposed along the proximal end portion of the lead body, and a plurality of conductors, each conductor of the plurality of conductors electrically coupling each of the plurality of terminals to at least one of the plurality of electrodes; and
disposing at least one anchoring unit along the distal end portion of the lead body, the at least one anchoring unit comprising a cylindrical lead attachment clement defining a central lumen within which a portion of the lead is received, and at least one at least one anchoring element this is disposed over the lead attachment element and extends away from the lead attachment element and is configured and arranged for contact with patient tissue to anchor the lead within the patient tissue.

16. The method of claim 15, wherein disposing at least one anchoring unit comprises sliding the at least one anchoring unit onto the distal end portion of the lead body.

17. The method of claim 16, wherein disposing at least one anchoring unit comprises swelling the at least one anchoring unit prior to sliding the at least one anchoring unit onto the distal end portion of the lead body.

18. The method of claim 15, wherein disposing at least one anchoring unit comprises adhesively attaching the at least one anchoring unit onto the distal end portion of the lead body.

19. The method of claim 15, further comprising ablating at least one region of the lead body to receive the at least one anchoring unit.

20. The method of claim 19, wherein ablating at least one region comprises pattering the at least one region of the lead body, wherein the at least one anchoring unit has a corresponding pattern on an interior surface thereof.
